(19) United States
(12) Patent Application Publication
(10) Pub. No.: US 2003/0096831 A1
(43) Pub. Date: May 22, 2003

(54) Ocular growth and nicotinic antagonists

(76) Inventors: Richard A. Stone, Havertown, PA (US); Jon M. Lindstrom, Radnor, PA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/194,494

(22) Filed: Jul. 10, 2002

Related U.S. Application Data
(63) Continuation of application No. PCT/US01/01692, filed on Jan. 18, 2001.
(60) Provisional application No. 60/176,875, filed on Jan. 18, 2000.

(51) Int. Cl. A61K 31/4747

(52) U.S. Cl. 514/278

(57) ABSTRACT

The invention relates to a method comprising the step of ocular administration of therapeutically effective amounts of a nicotinic antagonist to control postnatal ocular growth or inhibit the development of myopia.

### CROSS-REFERENCE TO RELATED APPLICATIONS

This application is a continuation of international application number PCT/US01/01692, and claims the benefit of priority of international application number PCT/US01/01692 having international filing date of Jan. 18, 2001, designating the United States of America and published in English, which claims the benefit of priority of U.S. provisional patent application No. 60/176,875, filed Jan. 18, 2000; both of which are hereby expressly incorporated by reference in their entireties.

### FIELD OF THE INVENTION

The present invention relates to the control of eye growth by nicotinic receptor antagonists, more particularly to the inhibition of postnatal ocular growth and the prevention of myopia in a host animal by ocular administration of nicotinic receptor antagonists.

### BACKGROUND OF THE INVENTION

Visual input dominates the regulation of post-natal eye growth and the development of refractive errors. Eye growth appears largely controlled locally in the eye, likely through the retina; specific roles for other components of the nervous system, such as the brain or peripheral nervous system, remain unclear (Stone, 1997, Myopia Updates: Proceedings of the 6th International Conference on Myopia, pp. 241-254; Wallman, 1993, Progress in Retinal Research, 12:133-153). As complex qualities of the visual image such as blur influence eye growth, it seems reasonable that neurons in the proximal retina might comprise the elements of a local regulatory mechanism. Indeed, much current evidence implicates several classes of retinal amacrine cells in the pathway linking visual input and eye growth control. The data most strongly supports a role for dopaminergic amacrine cells (Stone, 1997, Myopia Updates: Proceedings of the 6th International Conference on Myopia, pp. 241-254; Stone et al., 1989, Proc. Natl. Acad. Sci. U.S.A., 86:704-6). While evidence implicating other retinal neurons is either less fully developed or controversial, other subtypes of retinal amacrine cells hypothesized to influence refractive development include those containing vasoactive intestinal peptide (Pickett Seltner and Stell, 1995, Vision Res., 35:1265-1270; Stone et al., 1988, Proc. Natl. Acad. Sci. U.S.A., 85:257-60), glucagon (Fischer et al., 1999a, Nature Neuroscience, 2:706-12), nitric oxide (Fujikado et al., 1997, Curr. Eye Res., 16:992-6), enkephalin (Pickett Seltner et al., 1997, Vis. Neurosci., 14:801-809) and acetylcholine (Stone et al., 1991, Exp. Eye Res., 52:755-8).

Cholinergic mechanisms, acting through muscarinic receptors, seem involved in eye growth control because the muscarinic antagonist atropine retards the development of myopia in chick (Stone et al., 1991, Exp. Eye Res., 52:755-8), tree shrew (McKanna and Casagrande, 1981, Documenta Ophthalmologica Proceedings Series, 28:187-192), monkey (Raviola and Wiesel, 1985, N. Engl. J. Med., 312:1609-15; Tigges et al., 1999, Optometry & Vision Science, 76:397-407) and human (Brodstein et al., 1984, Ophthalmol., 91:1373-1379). Identifying the specific cholinergic neurons responsible for the regulation of eye growth, however, has proved difficult.

Because of the clinical association of myopia development with near work, it has long been hypothesized that accommodation underlies the mechanism causing myopia and that cycloplegia explains the anti-myopia activity of atropine, thus suggesting a role for the cholinergic neurons of the ciliary ganglion and the muscarinic receptors of the ciliary muscle. However, little experimental work supports a role for accommodation in the development of myopia despite many attractive features of the hypothesis. Ciliary ganglionectomy (Lin et al., 1996, Curr. Eye Res., 15:453-60; Raviola and Wiesel, 1985, N. Engl. J. Med., 312:1609-15) sectioning of the ciliary nerves (Shih et al., 1994, Invest. Ophthalmol. Vis. Sci., 35:3691-701) or lesioning the pre-ganglionic input to the ciliary ganglion at the Edinger-Westphal nucleus (Troilo, 1990, Ciba Foundation Symposium, 155:89-102; discussion 102-14) each fail to have a major impact on the development of experimental myopia. Pharmacologic evidence also argues against a role for accommodation in myopia development. An M1-selective muscarinic antagonist with minimal cycloplegic activity is at least as effective as atropine against experimental myopia in the rhesus monkey (Tigges et al., 1999, Optometry & Vision Science, 76:397-407). Further, it has been observed that the chick ciliary body contains striated rather than smooth muscle, that avian accommodation is controlled by nicotinic rather than muscarinic mechanisms, and that atropine fails to paralyze accommodation in the chick (Stone et al., 1991, Exp. Eye Res., 52:755-8). That atropine blocks myopia in the chick further argues against an accommodation mechanism (Stone, 1997, Myopia Updates: Proceedings of the 6th International Conference on Myopia, pp. 241-254).

Alternative cholinergic mechanisms have been sought to explain refractive development. Retinal cholinergic neurons and muscarinic receptors have been proposed, based on the activity of an M1-selective, but not M3-selective, muscarinic subtype receptor antagonists in chick myopia (Stone et al., 1991, Exp. Eye Res., 52:755-8). This notion is also supported by the anti-myopia activity of an M1-selective muscarinic antagonist in tree shrew (Cottriall and McBrien, 1996, Invest. Ophthalmol. Vis. Sci., 37:1368-79) and rhesus monkey (Tigges et al., 1999, Optometry & Vision Science, 76:397-407). However, in studies carried out to identify the cholinergic neurons involved, it was found that the activity of the biosynthetic enzyme for acetylcholine (choline acetyltransferase) was unaltered in the retina of myopic chick eyes but depressed in the ciliary ganglion (Pendrak et al., 1995, Exp. Eye Res., 60:237-43). Based upon responses of cultured chick scleral cells (Lind et al., 1998, Invest. Ophthalmol. Vis. Sci., 39:2217-2231) and the responses of the chick eye to retinal toxins that lesion cholinergic amacrine cells (Fischer et al., 1998b, Brain Res., 794:48-60), it has been suggested that muscarinic antagonists might inhibit myopia development by acting at extra-retinal sites such as sclera or choroid.

Further limiting our understanding the role of cholinergic neurons in eye growth control is the paucity of studies addressing cholinergic nicotinic mechanisms. Both intravitreal and subconjunctival nicotine induce accommodation in chicks (Reiner et al., 1995, Vision Res., 35:1227-1245). Twice daily intravitreal injections of nicotine for two weeks induce in chicks about a 2 diopter myopic shift in refraction compared to contralateral non-injected eyes; however, intravitreal saline injection had the same effect (Reiner et al., 1995, Vision Res., 35:1227-1245). Daily subconjunctival nicotine injections in chicks did cause a slight myopic refractive shift of 0.75 diopters compared to non-treated eyes, a response not seen for subconjunctival saline (Reiner et al., 1995, Vision Res., 35:1227-1245); but this degree of refractive shift in chicks may be of little biological significance because it approximates the focal depth of the chick eye (Schmid and Wildsoet, 1997, Ophthal. Physiol. Opt., 17:61-7). Nicotine's high lipophilicity would permit rapid diffusion from the eye, such that potential action at extra-ocular sites further limits mechanistic interpretation of these results. When vecuronium bromide, a neuromuscular blocking agent and nicotinic antagonist, was applied to chick corneas, it paralyzed accommodation but failed to influence the ocular elongation following spectacle-induced hyperopic defocus, again arguing against an accommodative mechanism for myopia (Schwahn and Schaeffel, 1994, Invest. Ophthalmol. Vis. Sci., 35:3516-24). Charged antagonists at the neuromuscular junction, of which d-tubocurarine is a prototype, typically penetrate poorly into the central nervous system and bind to all nicotinic receptor subtypes with low affinity (Gotti et al., 1997, Progress in Neurobiology, 53:199-237). Vecuronium bromide is also highly charged; thus, while it diffuses readily to block the neuromuscular junctions of intraocular muscles, it may not have access to receptor sites in lipophilic tissues potentially involved in eye growth control, such as the neural retina.

Besides the nicotinic acetylcholine receptors at the neuromuscular junctions of the intraocular muscles, the chick eye also has well-characterized nicotinic receptor subtypes in both retina (Hamassaki-Britto et al., 1994a, Vis. Neurosci., 11:63-70; Hamassaki-Britto et al., 1994b, J. Comp. Neurol., 347:161-170; Keyser et al., 1993, J. Neurosci., 13:442-454; Vailate et al., 1999, Mol. Pharmacol., 56:11-19) and ciliary ganglion (Berg et al., 1998, Neuronal Nicotinic Receptors: Pharmacology and Therapeutic Opportunities, pp. 187-196; Conroy and Berg, 1995, J. Biol. Chem., 270:4424-4431; Halvorsen and Berg, 1990, J. Neurosci., 10:1711-1718; Horch and Sargent, 1995, J. Neurosci., 15:7778-7795; Pugh et al., 1995, Mol. Pharmacol., 47:717-725).

### SUMMARY OF THE INVENTION

The invention concerns the use of nicotinic antagonists of suitable solubility to penetrate to the relevant targets that regulate postnatal eye growth and that inhibit postnatal ocular growth and the development of myopia.

The invention provides a method of controlling postnatal ocular growth by ocular administration of therapeutically effective amounts of a nicotinic antagonist to control postnatal ocular growth. Further provided is a method of inhibiting the abnormal postnatal axial growth of the eye of a host animal by administering therapeutically effective amounts of a nicotinic antagonist during postnatal development. The invention also provides a method of inhibiting abnormal equatorial expansion of the eye of a host animal by administering therapeutically effective amounts of a nicotinic antagonist during postnatal development. The invention further provides a method of inhibiting the abnormal vitreous cavity expansion of the eye of a host animal by administering therapeutically effective amounts of a nicotinic antagonist during postnatal development. Another aspect of the invention provides a method of preventing or inhibiting development of myopia by ocular administration of therapeutically effective amounts of a nicotinic antagonist.

The invention provides for the use of a nicotinic antagonist for the preparation of a medicament adapted for ocular administration for the control of postnatal ocular growth. The invention further provides for the use of a nicotinic antagonist for the preparation of a medicament for uses such as inhibiting the abnormal axial growth of the eye of a host animal during postnatal development, inhibiting the abnormal equatorial expansion of the eye of a host animal during postnatal development. inhibiting the abnormal vitreous cavity expansion of the eye of a host animal during postnatal development. The invention also provides for the use of a nicotinic antagonist for the preparation of a medicament adapted for ocular administration for the prevention or treatment of myopia.

In one embodiment of the invention, the nicotinic antagonist may be a competitive nicotinic antagonist such as methyllcaconitine or dihydro-β-erythroidine. In another embodiment, the nicotinic antagonist may be a channel-blocking nicotinic antagonist such as chlorisondamine or mecamylamine. In a further embodiment of the invention, the nicotinic antagonist may be a noncompetitive nicotinic antagonist such as sertraline, paroxetine, nefaxodone, venlafaxine, fluoxetine, buproprion, phencyclidine, and ibogaine. In another embodiment of the invention, the nicotinic antagonist may be an antibody inhibiting nicotinic receptor function. In yet another embodiment of the invention, the nicotinic antagonist may be an agonist that acts like a nicotinic antagonist.

The invention provides method of detecting the ability of a nicotinic antagonist to control postnatal ocular growth of the eye of a host animal by contacting an animal eye with a therapeutically effective amount of a nicotinic antagonist, detecting the change in growth of the eye exposed to a therapeutically effective amount of a nicotinic antagonist, then applying a known control agent in a second eye, observing the results of the control agent on the change in growth of the second eye, and comparing the change in growth of the first eye exposed to a therapeutically effective amount of a nicotinic antagonist with the change in growth of the second eye exposed to a known control agent, thereby identifying the nicotinic antagonist as having the ability to control postnatal ocular growth. Further provided is a method of making a pharmaceutical including the steps of identifying a nicotinic antagonist as an active agent having the ability to control postnatal ocular growth and combining the active agent in admixture with a pharmaceutical excipient.

The invention provides a method of identifying compounds that can be used to modulate myopia including the steps of incubating a cell that expresses a nicotinic receptor in the presence and absence of a test compound, determining whether the test compound binds to at least one nicotinic receptor, selecting a test compound that binds to at least one nicotinic receptor, administering the selected test compound to a test animal, determining whether the test compound alters the development of myopia of the test animal, and selecting a compound that alters the development of myopia of a test animal.

### BRIEF DESCRIPTION OF THE DRAWINGS

FIG. 1 shows drug effects on refractions of goggled eyes. Chlorisondamine (CHL; P<0.001), mecamylamine (MEC; P<0.001) and methyllycaconitine (MLA; P=0.04) each influenced the myopic refraction occurring beneath a goggle, as assessed by ANOVA; but dihydro-β-erythroidine (DHBE) had no effect on the refraction of visually deprived eyes. For the results of pairwise comparisons by the Tukey test, see Table 1. The number of chicks in each experimental group is indicated here. Data are shown as the difference between the goggled and contralateral open eye (mean±S.E.M.). To facilitate comparisons, the bar for each control group is cross-hatched.

FIG. 2, A-D, shows drug effects on ocular dimensions of goggled eyes. FIG. 2A shows drug effects on axial length measured by ultrasound. FIG. 2B shows drug effects on vitreous cavity length measured by ultrasound. FIG. 2C shows drug effects on axial length measured by digital calipers. FIG. 2D shows drug effects on equatorial diameter measured by calipers. Chlorisondamine (CHL), mecamylamine (MEC) and methyllycaconitine (MLA) each reduced the excessive ocular growth occurring beneath a goggle; the statistically significant effects (P<0.05) and suggestive trends are identified by the ANOVA results directly on each data set. The effects of dihydro-β-erythroidine (DHBE) were weaker as only the axial measurements by calipers showed a statistically significant drug effect. For the results of pairwise comparisons by the Tukey test, see Table 1. The number of chicks in each experimental group is provided in FIG. 1. Data are shown as the difference between the goggled and contralateral open eye (mean±S.E.M.). To facilitate comparisons, the bar for each control group is cross-hatched.

FIG. 3 shows effects of chlorisondamine on non-goggled eyes. Unilateral administration of chlorisondamine to eyes of never-goggled chicks shifted overall refraction towards hyperopia (ANOVA on ranks: P=0.004), reduced axial length (ANOVA on ranks: ultrasound, P=0.03; calipers, P=0.03) and inhibited the axial expansion of the vitreous cavity (ANOVA on ranks: P=0.01). For the results of pairwise comparisons by the Tukey test, see Table 1. N=9-20 chicks per group. The data are illustrated as the difference between the drug treated and contralateral vehicle treated eye (mean±S.E.M.).

Table A presents a summary of nicotinic receptor subtypes.

Table 1 shows post hoc pairwise comparisons of drug effects using the Tukey test

Table 2 shows the longer term effects of a single-dose of chlorisondamine (200 μg) on form deprivation myopia.

### DETAILED DESCRIPTION OF THE INVENTION

The nervous system, in large part through the retina, controls eye growth postnatally, and the development of refractive errors (the need for glasses) appears to be chiefly dependent on neural mechanisms. The most common refractive error clinically is myopia. The present disclosure includes a pharmaceutical drug class, nicotinic antagonists, with activity against an experimental model of myopia, but which in addition also inhibits “normal” eye growth.

In the present invention, the inventors tested the effects on eye chick growth of several nicotinic antagonists with favorable pharmacologic properties, using the efficient delivery route of intravitreal injection. Four nicotinic antagonists selected were chlorisondamine (CHL), mecamylamine (MEC), methyllcaconitine (MLA) and dihydro-β-erythroidine (DHBE). Animal models using chicks offer advantages of good optics as a model for the human eye. Using antagonists with established profiles against neuronal nicotinic receptors and having lipophilic properties compatible with diffusion into neural tissue, we found evidence for an actual role, perhaps a central role, of nicotinic receptors in control of ocular growth.

The invention is directed to the use of nicotinic antagonists having suitable solubility properties to penetrate to the relevant target sites that regulate postnatal eye growth and that inhibit postnatal ocular growth or the development of myopia. All drug classes previously identified for controlling eye growth show activity only against the form deprivation myopia model (eye remains covered by a goggle beginning at approximately 1 week of age). The present invention identifies a drug class that is also active against open eyes (vision has not been deprived by goggles during maturation); and it is the first class of agents identified to show this activity. This activity against open eyes is advantageous in application to human myopia where form deprivation is not the usual circumstance. While chicks utilize nicotinic receptors in the control of pupil size and accommodation, mammalian eyes use a subclass of muscarinic receptors to control pupil size and accommodation; therefore, nicotinic antagonists are expected to be well tolerated following local application in the human eye, without inducing pupil dilation and paralysis of accommodation in children.

Section 1. Nicotinic Receptor Subtypes

Nicotinic acetylcholine receptors subtypes are composed of five homologous subunits that form an acetylcholine-gated cation channel (Lindstrom, 1997, Mol. Neurobiology, 15:193-222). There are 17 known nicotinic receptor subunits (α1-10, β1-4, γ, δ, and ε). Each subunit possesses four transmembrane domains. The acetylcholine binding site is formed by at least three peptide loops on the α-subunit (principal component), and two on the adjacent subunit (complementary component). All α-subunits possess two tandem cysteine residues near to the site involved in acetylcholine binding, and subunits not named a lack those tandem cysteines. Alternatively spliced forms of the α4 subunit (α4-1 and α4-2; rat) and α1 subunit (human) have been identified. (Mandelzys, A. et al. (1995) J. Neurophysiol. 74, 1212-1221; Papke, R. L. et al. (1996) Neurosci. Lett. 213, 201-204; Albuquerque, E. X. et al. (1997) J. Pharmacol. Exp. Ther. 280, 1117-1136; deFiebre, C. M. et al. (1995) Mol. Pharmacol. 47, 164-171).

The receptors fall into three general classes: a muscle class and two neural classes. The muscle types exist in only two forms—a fetal and an adult form, each with al subunits and other subunits specific for muscle receptors. One class of neuronal receptors binds α-bungarotoxin and is composed of α7, α8, α9 or α10 subunits, often as homomeric receptors. The other class of neural receptors does not bind a-bungarotoxin and is formed from combining α2, α3, α4 or α6 subunits with β2 or β4 subunits. Rapid desensitization and limited availability of selective drugs suited for in vivo studies have impaired defining physiologic functions for these biochemically defined receptor subtypes. A summary of nicotinic receptor subtypes is presented in Table A.

The nicotinic receptor subcommittee of NC-IUPHAR has recommended a nomemclature and classification scheme for nicotinic acetylcholine (nACh) receptors based on the subunit composition of known, naturally-expressed nACh receptor subtypes and/or on subtypes formed by heterologous expression (Lukas et al. (1999) IUPHAR XX. “Current status of the nomenclature for nicotinic acetylcholine receptors and their subunits,” Pharm. Rev. 51, 397-401). Headings for Table A reflect abbreviations designating nACh receptor subtypes based on the predominant α subunit contained in that receptor subtype. An asterisk following the indicated α subunit means that other subunits are known to or might assemble with the indicated α subunit to form the designated nACh receptor subtype(s). The absence of an asterisk indicates that the indicated subunit is known to assemble into a homomeric nACh receptor subtype. Where subunit stoichiometries are known, numbers of a particular subunit in a specific nACh receptor subtype are indicated by a subscript following the subunit in brackets. All subunits are of mammalian origin with the exception of α8 (avian).

Section 1.1. Nicotinic Receptor Subtypes and the Eye.

The chick retina contains several classes of cholinergic neurons (Miller et al., 1987, Neurosci., 21:725-743). Besides several subtypes of muscarinic acetylcholine receptors (Fischer et al., 1998a, J. Comp. Neurol., 392:273-84), the chick retina expresses a multiplicity of nicotinic acetylcholine receptor subunits, including α3, α6, α7, α8 and β2, β3, β4. The cellular patterns of neural localization of nicotinic receptors in chick retina are complex (Hamassaki-Britto et al., 1994a, Vis. Neurosci., 11:63-70; Hamassaki-Britto et al., 1994b, J. Comp. Neurol., 347:161-170; Keyser et al., 1993, J. Neurosci., 13:442-454; Vailate et al., 1999, Mol. Pharmacol., 56:11-19). The chick ciliary ganglion is similarly enriched with a diversity of nicotinic receptor subtypes that include the α3 subunits that typifies autonomic ganglia, α5, α7, β2 or β4 subunits, with both synaptic and extrasynaptic localizations (Berg et al., 1998, Neuronal Nicotinic Receptors: Pharmacology and Therapeutic Opportunities, pp. 187-196; Conroy and Berg, 1995, J. Biol. Chem., 270:4424-4431; Horch and Sargent, 1995, J. Neurosci., 15:7778-7795; Pugh et al., 1995, Mol. Pharmacol., 47:717-725).

Section 2. Nicotinic Antagonists.

The function of nicotinic acetycholine receptors (nicotinic receptors, or NR) can be antagonized by various compounds. The action of these compounds against NR may be complex, may involve more than one mechanism of antagonism, and may not yet have been fully characterized in all details. Specific drugs for discussion purposes are classified in terms of their currently best understood mechanism of action. Nicotinic antagonists are defined as compounds that inhibit, block, compete, prevent, or otherwise interfere with any effect of a nicotinic agonist on a target. We claim use of competitive nicotinic antagonists in this invention. Competitive nicotinic antagonists are defined as compounds that appear to compete for the agonist binding sites on nicotinic receptors, where competitive antagonists appear to inhibit receptor function by preventing activation of the receptor by agonists. Examples of competitive antagonists may include, but are not limited to, dihydro-β-erythroidine, bungarotoxins, tubocurarine, methyllcaconitine, the peptide conotoxins derived from snails, including MI, EI, GI, SI, SIA, SII, as well as other naturally occurring peptide antagonists and synthetic peptide antagonists derived from expression libraries (Lindstrom, 1997, Mol. Neurobiology, 15:193-222).

We claim use of channel-blocking nicotinic antagonists in this invention. Channel-blocking nicotinic antagonists are defined as compounds that appear to block the ion channel of nicotinic receptors (NR), thereby preventing the transmembrane ion flux required for nicotinic receptor function. Examples of channel-blocking nicotinic antagonists may include, but are not limited to, chlorisondamine, mecamylamine, hexamethonium, amantadine, memantine, dizocilpine [(+)-MK-801], 8 (dethylamino)octyl-3,4,5-trimethoxybenzoate (TMB-8), and zinc. (Bencherif et al., 1995, J. Pharmacol. and Exper. Therapeutics, 275: 1418-1426; Buisson and Bertrand, 1998, Mol. Pharmacol., 53: 555-563). While these compounds appear to act preferentially on open channels (Peng et al., 1997, Mol. Pharmacol., 51: 776-784; Buisson and Bertrand, 1998, Mol. Pharmacol., 53: 555-563), compounds that block closed channels are also suitable for use in this invention.

We claim use of noncompetitive nicotinic antagonists in this invention. Noncompetitive nicotinic antagonists are defined as compounds that antagonize the functions of nicotinic receptors (NR), but do not appear to block the ligand binding site or directly block the ion channel. Functional blockade by a noncompetitive nicotinic antagonist of ion flux through the ion channel of nicotinic receptors is insurmountable by increasing agonist concentration (Fryer and Lukas, 1999a, J. Pharmacol. and Exper. Therapeutics, 288: 88-92; Fryer and Lukas, 1999b, J. Neurochem., 72: 1117-1124). Ethanol and volatile anesthetics including tetracaine and procaine are noncompetitive functional nicotinic antagonists for diverse nicotinic receptor subtypes (Bencherif et al., 1995, J. Pharmacol. and Exper. Therapeutics, 275: 1418-1426; Lindstrom, 1997, Mol. Neurobiology, 15:193-222). Unexpected noncompetitive nicotinic antagonists include psychoactive compounds such as buproprion, phencyclidine, ibogaine, sertraline, paroxetine, nefaxodone, venlafaxine, and fluoxetine (Fryer and Lukas, 1999a, J. Pharmacol. and Exper. Therapeutics, 288: 88-92; Fryer and Lukas, 1999b, J. Neurochem., 72: 1117-1124). Other noncompetitive nicotinic antagonists may act as negative allosteric effectors, acting via allosteric sites used by known postive allosteric effectors such as ivermectin, or acting on distinct sites on the receptor (Krause et al., 1998, Mol. Pharmacol., 53: 283-294). Certain voltage-dependent mechanisms can also function as noncompetitive antagonists, including voltage-sensitive Mg²⁺ block of nicotinic receptor ion channels, voltage-dependent channel blockage by intracellular spermine, and nicotinic receptor inactivation triggered by membrane depolarization.

We claim use of antibodies that act as nicotinic antagonists in this invention. Antibodies can also act as nicotinic antagonists; for example, the monoclonal antibody mAb 319 blocks the function of nicotinic receptors (NR) when injected into cells (Cuevas and Berg, 1998, J. Neurosci. 18: 10335-10344). Antibodies include polyclonal antibodies, monoclonal antibodies, humanized or chimerized antibodies, single chain antibodies, FAb fragments F(Ab)′₂ fragments, fragments produced by a FAb expression library, anti-idiotypic (anti-Id) antibodies, and epitope-binding fragments of any of the above.

We claim use of agonists that function as nicotinic antagonists in this invention. Agonists can also function as nicotinic antagonists under certain circumstances, for example, based on their time-averaged antagonist effects. Reversible desensitization is observed following stimulation by all agonists including, but not limited to, acetylcholine, nicotine, epibatidine, cytisine, methylcarbamylcholine, and DMPP. Some compounds have a bifunctional effect, acting as an agonist for some nicotinic receptor subtypes and as an antagonist for others. The heterocyclic substituted pyridine derivative (+/−)-2-(-3-pyridinyl)-1-azabicyclo [2.2.2]octane, also known as RJR-2429, selectively activates human muscle nicotinic receptors and a putative α3β4-containing receptor, but inhibits nicotinic receptors in preparations of rat thalamus. This compound is a partial agonist on nicotinic receptors mediating dopamine release from rat synaptosomal preparations. (Bencherif et al., 1998, J. Pharmacol. and Exper. Therapeutics, 284: 886-894). At sufficiently high concentrations, any agonist can cause reversible and irreversible desensitization and/or inactivation of nicotinic receptors. Exposure to high agonist concentrations thus give rise to time-averaged antagonist effects on the exposed cell. By way of example, this phenomenon is observed following chronic nicotine exposure at concentrations comparable to circulating nicotine levels found in human tobacco smokers, and can lead to inactivation of certain nicotinic receptor subtypes (Lindstrom, 1997, Mol. Neurobiology, 15:193-222).

Section 3. Screening Assays for Compounds that Modulate Nicotinic Receptor Activity

The following assays are designed to identify compounds that interact with nicotinic receptors (NR) that control postnatal eye growth, compounds that interfere with NR, and compounds which modulate the activity of NR genes or modulate the levels of NR. Assays may additionally be utilized which identify compounds which bind to NR and which may modulate NR levels.

The compounds which may be screened in accordance with the invention include, but are not limited to peptides, antibodies and fragments thereof, and other organic compounds (e.g., peptidomimetics) that bind to NR and either mimic the activity triggered by the natural ligand (i.e., agonists) or inhibit the activity triggered by the natural ligand (i.e., antagonists), as well as peptides, antibodies or fragments thereof, and other organic compounds that mimic a domain of the NR (or a portion thereof) and bind to and “neutralize” natural ligand.

Such compounds may include, but are not limited to, naturally occurring peptides such as conotoxins, synthetic peptides such as, for example, soluble peptides, including but not limited to members of random peptide libraries, (see, e.g., Lam et al., 1991, Nature, 354:82-84; Houghten et al., 1991, Nature, 354:84-86), and combinatorial chemistry-derived molecular library made of D- and/or L-configuration amino acids, phosphopeptides (including, but not limited to, members of random or partially degenerate, directed phosphopeptide libraries; (see, e.g., Songyang et al., 1993, Cell, 72:767-778), antibodies (including, but not limited to, polyclonal, monoclonal, humanized, anti-idiotypic, chimeric or single chain antibodies, and FAb, F(Ab′)₂ and FAb expression library fragments, and epitope-binding fragments thereof), and small organic or inorganic molecules.

Other compounds which can be screened in accordance with the invention include but are not limited to small organic molecules that are able to cross the blood-retinal or blood-aqueous humor barrier, gain entry into an appropriate cell and affect the expression of the NR gene or some other gene involved in the NR signal transduction pathway (e.g., by interacting with the regulatory region or transcription factors involved in gene expression); or such compounds that affect the activity of the NR or the activity of some other intracellular factor involved in the NR signal transduction pathway.

Computer modelling and searching technologies permit identification of compounds, or the improvement of already identified compounds, that can modulate NR activity. Having identified such a compound or composition, the active sites or regions are identified. Such active sites might typically be ligand binding sites, such as the interaction domains of ligands with NR itself. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modelling can be used to complete the structure or improve its accuracy. Any recognized modelling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. (Bencherif et al., 1998, J. Pharmacol. and Exper. Therapeutics, 284: 886-894). The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential NR modulating compounds.

Alternatively, these methods can be used to identify improved modulating compounds from an already known modulating compound or ligand. The composition of the known compound can be modified and the structural effects of modification can be determined using experimental and computer modelling methods such as those described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the active sites of NR, and related transduction and transcription factors will be apparent to those of skill in the art.

Examples of molecular modelling systems are the CHARMM and QUANTA programs (Polygen Corporation, Waltham, Mass.). CHARMM performs the energy minimization and molecular dynamics functions. QUANTA performs the construction, graphic modelling and analysis of molecular structure. QUANTA allows interactive construction, modification, visualization, and analysis of the behavior of molecules with each other.

A number of articles review computer modelling of drug interactions with specific proteins, such as Rotivinen et al., 1988, Acta Pharmaceutical Fennica 97:159-166; Ripka, New Scientist 54-57 (Jun. 16, 1988); McKinaly and Rossmann, 1989, Annu. Rev. Pharmacol. Toxiciol. 29:111-122; Perry and Davies, OSAR: Quantitative Structure-Activity Relationships in Drug Design pp. 189-193 (Alan R. Liss, Inc. 1989); Lewis and Dean, 1989, Proc. R. Soc. Lond. 236:125-140 and 141-162; and, with respect to a model receptor for nucleic acid components, Askew et al., 1989, J. Am. Chem. Soc. 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA encoding NR, once that region is identified.

Although described above with reference to design and generation of compounds which could alter binding, one could also screen libraries of known compounds, including natural products or synthetic chemicals, and biologically active materials, including proteins, for compounds which are inhibitors or activators.

Compounds identified via assays such as those described herein may be useful, for example, in elaborating the biological function NR, and for ameliorating myopia. Assays for testing the effectiveness of compounds, identified by, for example, techniques such as those described in Section 3.1 through 3.3, are discussed, below, in Section 3.4.

Section 3.1. In vitro Screening Assays for Compounds that Bind to NR

In vitro systems may be designed to identify compounds capable of interacting with (e.g., binding to) NR. Compounds identified may be useful, for example, in modulating the activity of wild type and/or mutant NR gene products; may be useful in elaborating the biological function of NR; may be utilized in screens for identifying compounds that disrupt normal NR interactions; or may in themselves disrupt such interactions.

The principle of the assays used to identify compounds that bind to NR involves preparing a reaction mixture of the NR and the test compound under conditions and for a time sufficient to allow the two components to interact and bind, thus forming a complex which can be removed and/or detected in the reaction mixture. The NR species used can vary depending upon the goal of the screening assay. For example, where agonists of the natural ligand are sought, the full length NR, or a truncated NR, a peptide corresponding to the extracellular domain or a fusion protein containing the NR ligand binding site fused to a protein or polypeptide that affords advantages in the assay system (e.g., labeling, isolation of the resulting complex, etc.) can be utilized. Where compounds that interact with the cytoplasmic domain (CD) are sought to be identified, peptides corresponding to the NR CD and fusion proteins containing the NR CD can be used.

The screening assays can be conducted in a variety of ways. For example, one method to conduct such an assay would involve anchoring the NR protein, polypeptide, peptide or fusion protein or the test substance onto a solid phase and detecting NR/test compound complexes anchored on the solid phase at the end of the reaction. In one embodiment of such a method, the NR reactant may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for NR protein, polypeptide, peptide or fusion protein or the test compound to anchor any complexes formed in solution, and a labeled antibody specific for the other component of the possible complex to detect anchored complexes.

Alternatively, cell-based assays can be used to identify compounds that interact with NR. To this end, cell lines that express NR, or cell lines that have been genetically engineered to express NR (e.g., by transfection or transduction of NR DNA) can be used. Interaction of the test compound with, for example, the heterologous NR expressed by the host cell can be determined by comparison or competition with native ligands.

Section 3.2. Assays for Intracellular Proteins that Interact with the NR

Any method suitable for detecting protein-protein interactions may be employed for identifying transmembrane proteins or intracellular proteins that interact with NR. Among the traditional methods which may be employed are co-immunoprecipitation, crosslinking and co-purification through gradients or chromatographic columns of cell lysates or proteins obtained from cell lysates and the NR to identify proteins in the lysate that interact with the NR. For these assays, the NR component used can be a full length NR, a soluble derivative lacking the membrane-anchoring region (e.g., a truncated NR in which the transmembrane region is deleted resulting in a truncated molecule containing the extracellular domain fused to the cellular domain), a peptide corresponding to the cellular domain or a fusion protein containing the cellular domain of NR. Once isolated, such an intracellular protein can be identified and can, in turn, be used, in conjunction with standard techniques, to identify proteins with which it interacts. For example, at least a portion of the amino acid sequence of an intracellular protein which interacts with the NR can be ascertained using techniques well known to those of skill in the art, such as via the Edman degradation technique. (See, e.g., Creighton, 1983, Proteins: Structures and Molecular Principles, pp.34-49). The amino acid sequence obtained may be used as a guide for the generation of oligonucleotide mixtures that can be used to screen for gene sequences encoding such intracellular proteins. Screening may be accomplished, for example, by standard hybridization or PCR techniques. Techniques for the generation of oligonucleotide mixtures and the screening are well-known. (See, e.g., PCR Protocols: A Guide to Methods and Applications, 1990).

Additionally, methods may be employed which result in the simultaneous identification of genes which encode the transmembrane or intracellular proteins interacting with NR. These methods include, for example, probing expression libraries, in a manner similar to the well known technique of antibody probing of λgt11 libraries, using labeled NR protein, or an NR polypeptide, peptide or fusion protein, e.g., an NR polypeptide or NR domain fused to a marker (e.g., an enzyme, fluor, luminescent protein, or dye), or an IgG-Fc domain.

One method which detects protein interactions in vivo, the two-hybrid system, is described in detail for illustration only and not by way of limitation. One version of this system has been described (Chien et al., 1991, Proc. Natl. Acad. Sci. USA, 88:9578-9582) and is commercially available from Clontech (Palo Alto, Calif.).

Briefly, utilizing such a system, plasmids are constructed that encode two hybrid proteins: one plasmid consists of nucleotides encoding the DNA-binding domain of a transcription activator protein fused to a nucleotide sequence encoding NR, an NR polypeptide, peptide or fusion protein, and the other plasmid consists of nucleotides encoding the transcription activator protein's activation domain fused to a cDNA encoding an unknown protein which has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the hybrid containing the DNA-binding domain cannot activate transcription because it does not provide activation function, and the hybrid containing the activation domain cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, NR may be used as the bait gene product. Total genomic or cDNA sequences are fused to the DNA encoding an activation domain. This library and a plasmid encoding a hybrid of a bait NR gene product fused to the DNA-binding domain are cotransformed into a yeast reporter strain, and the resulting transformants are screened for those that express the reporter gene. For example, and not by way of limitation, a bait NR gene sequence, such as the open reading frame can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line used to detect proteins that interact with bait NR gene product, can be made using methods routinely practiced in the art. According to the particular system described herein, for example, the cDNA fragments can be inserted into a vector such that they are translationally fused to the transcriptional activation domain of GALA. This library can be co-transformed along with the bait NR gene-GALA fusion plasmid into a yeast strain which contains a lacZ gene driven by a promoter which contains GAL4 activation sequence. A cDNA encoded protein, fused to GAL4 transcriptional activation domain, that interacts with bait NR gene product will reconstitute an active GAL4 protein and thereby drive expression of the HIS3 gene. Colonies which express HIS3 can be detected by growth on petri dishes containing semi-solid agar based media lacking histidine. The cDNA can then be purified from these strains, and used to produce and isolate the protein that interacts with the bait NR gene product using techniques routinely practiced in the art.

Section 3.3. Assays for Compounds that Interfere with NR/Intracellular or NR/Transmembrane Macromolecule Interaction

The macromolecules that interact with the NR are referred to, for purposes of this discussion, as “binding partners”. These binding partners are likely to be involved in the NR signal transduction pathway, and therefore, in the role of NR in controlling postnatal ocular growth. Therefore, it is desirable to identify compounds that interfere with or disrupt the interaction of such binding partners with NR which may be useful in regulating the activity of NR and control of postnatal ocular growth associated with NR activity.

The basic principle of the assay systems used to identify compounds that interfere with the interaction between the NR and its binding partner or partners involves preparing a reaction mixture containing NR protein, polypeptide, peptide or fusion protein as described in Sections 3.1 and 3.2 above, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of the NR moiety and its binding partner. Control reaction mixtures are incubated without the test compound, or with a placebo. The formation of any complexes between the NR moiety and the binding partner is then detected. The formation of a complex in the control reaction, but not in the reaction mixture containing the test compound, indicates that the compound interferes with the interaction of the NR and the interactive binding partner. Additionally, complex formation within reaction mixtures containing the test compound and normal NR protein may also be compared to complex formation within reaction mixtures containing the test compound and a mutant NR. This comparison may be important in those cases wherein it is desirable to identify compounds that disrupt interactions of mutant but not normal NRs.

The assay for compounds that interfere with the interaction of the NR and binding partners can be conducted in a heterogeneous or homogeneous format. Heterogeneous assays involve anchoring either the NR moiety product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction by competition can be identified by conducting the reaction in the presence of the test substance; i.e., by adding the test substance to the reaction mixture prior to or simultaneously with the NR moiety and interactive binding partner. Alternatively, test compounds that disrupt preformed complexes, e.g. compounds with higher binding constants that displace one of the components from the complex, can be tested by adding the test compound to the reaction mixture after complexes have been formed. The various formats are described briefly below.

In a heterogeneous assay system, either the NR moiety or the interactive binding partner is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the NR or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the NR moiety and the interactive binding partner is prepared in which either NR or its binding partners is labeled, but the signal generated by the label is quenched due to formation of the complex (see, e.g., U.S. Pat. No. 4,109,496 by Rubenstein, which utilizes this approach for immunoassays). The addition of a test substance that competes with and displaces one of the species from the preformed complex will result in the generation of a signal above background. In this way, test substances which disrupt the interaction between NR and an intracellular binding partner can be identified.

In a particular embodiment, an NR fusion can be prepared for immobilization. For example, NR or a peptide fragment can be fused to a glutathione-S-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art. This antibody can be labeled with ¹²⁵I, for example, by methods routinely practiced in the art. In a heterogeneous assay, e.g., the GST-NR fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between NR (as a gene product) and the interactive binding partner can be detected by measuring the amount of radioactivity that remains associated with the glutathione-agarose beads. A successful inhibition of the interaction by the test compound will result in a decrease in measured radioactivity.

Alternatively, the GST-NR fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the NR/binding partner interaction can be detected by adding the labeled antibody and measuring the radioactivity associated with the beads.

In another embodiment of the invention, these same techniques can be employed using peptide fragments that correspond to the binding domains of the NR and/or the interactive or binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the intracellular binding partner is obtained, short gene segments can be engineered to express peptide fragments of the protein, which can then be tested for binding activity and purified or synthesized.

For example, and not by way of limitation, a GST-NR fusion protein can be prepared and anchored to a solid material as described, for example by allowing it to bind to glutathione agarose beads. The interactive binding partner can be labeled with a label, such as ³⁵S, and cleaved with a proteolytic enzyme such as trypsin. Cleavage products can then be added to the anchored GST-NR fusion protein and allowed to bind. After washing away unbound peptides, labeled bound material, representing the intracellular binding partner binding domain, can be eluted, purified, and analyzed for amino acid sequence by well-known methods. Peptides so identified can be produced synthetically or fused to appropriate facilitative proteins using recombinant DNA technology.

Section 3.4. Assays for Identification of Compounds that Ameliorate Abnormal Postnatal Ocular Growth

Compounds, including but not limited to binding compounds identified via assay techniques such as those described, above, in Sections 3.1 through 3.3, can be tested for the ability to ameliorate abnormal postnatal ocular growth, including myopia. The assays described above can identify compounds which affect NR activity (e.g., compounds that bind to the NR, inhibit binding of the natural ligand, and either activate signal transduction (agonists) or block activation (antagonists), and compounds that bind to the natural ligand of the NR and neutralize ligand activity); or compounds that affect NR gene activity (by affecting NR gene expression, including molecules, e.g., proteins or small organic molecules, that affect or interfere with splicing events so that expression of the full length or the truncated form of the NR can be modulated). However, it should be noted that the assays described can also identify compounds that modulate NR signal transduction (e.g., compounds which affect downstream signalling events, such as inhibitors or enhancers of tyrosine kinase or phosphatase activities which participate in transducing the signal activated by ligand binding to the NR). The identification and use of such compounds which affect another step in the NR signal transduction pathway in which the NR gene and/or NR gene product is involved, and by affecting this same pathway may modulate the effect of NR on the development of abnormal postnatal ocular growth, are within the scope of the invention. Such compounds can be used as part of a therapeutic method for the treatment of myopia and other conditions resulting from abnormal postnatal ocular growth.

The invention encompasses cell-based and animal model-based assays for the identification of compounds exhibiting such an ability to ameliorate myopia symptoms, signs or characteristics. Such cell-based assay systems can also be used as the “gold standard” to assay for purity and potency of natural ligands, including recombinantly or synthetically produced ligands.

Cell-based systems can be used to identify compounds which may act to ameliorate myopia symptoms, signs or characteristics. Such cell systems can include, for example, recombinant or non-recombinant cells, such as cell lines, which produce NR. For example, retinal cells or cell lines derived from retina can be used. In addition, expression host cells (e.g., COS cells, CHO cells, fibroblasts) genetically engineered to express a functional NR and to respond to activation by the natural ligand, e.g., as measured by a chemical or phenotypic change, induction of another host cell gene, change in ion flux (e.g., Na⁺, K⁺), tyrosine phosphorylation of host cell proteins, etc., can be used as an end point in the assay.

In utilizing such cell systems, cells may be exposed to a compound suspected of exhibiting an ability to ameliorate myopia symptoms in intact eyes, at a sufficient concentration and for a time sufficient to elicit amelioration of myopia-related cellular phenotypes or cell functions in the exposed cells. After exposure, the cells can be assayed to measure alterations in gene expression, e.g., by assaying cell lysates for mRNA transcripts (e.g., by Northern analysis) or for NR protein expressed in the cell; compounds which regulate or modulate expression of the NR gene are good candidates as therapeutics. Alternatively, the cells are examined to determine whether one or more myopia-related cellular phenotypes or cell functions has been altered to resemble a more normal or more wild type, non-myopic phenotype, or a phenotype more likely to produce a lower incidence or severity of myopia symptoms. Still further, the expression and/or activity of components of the signal transduction pathway of which NR is a part, or the activity of the NR signal transduction pathway itself can be assayed.

For example, after exposure, the cell lysates can be assayed for the presence of tyrosine phosphorylation of host cell proteins, as compared to lysates derived from unexposed control cells. The ability of a test compound to inhibit tyrosine phosphorylation of host cell proteins in these assay systems indicates that the test compound inhibits signal transduction initiated by NR activation. The cell lysates can be readily assayed using a Western blot format; i.e., the host cell proteins are resolved by gel electrophoresis, transferred and probed using a anti-phosphotyrosine detection antibody (e.g., an anti-phosphotyrosine antibody labeled with a signal generating compound, such as radiolabel, fluor, enzyme, etc.) (See, e.g., Glenney et al., 1988, J. Immunol. Methods, 109:277-285; Frackelton et al., 1983, Mol. Cell. Biol., 3:1343-1352). Alternatively, an ELISA format could be used in which a particular host cell protein involved in the NR signal transduction pathway is immobilized using an anchoring antibody specific for the target host cell protein, and the presence or absence of phosphotyrosine on the immobilized host cell protein is detected using a labeled anti-phosphotyrosine antibody. (See, King et al., 1993, Life Sciences, 53:1465-1472). In yet another approach, ion flux, such as sodium or potassium ion flux, can be measured as an end point for NR stimulated signal transduction. Membrane depolarization can also be measured as an end point for NR stimulated effects.

In addition, animal-based myopia models may be used to identify compounds capable of ameliorating myopia-like symptoms. Such animal models may be used as test substrates for the identification of drugs, pharmaceuticals, therapies and interventions which may be effective in treating such disorders. For example, animal models may be exposed to a compound suspected of exhibiting an ability to ameliorate myopia symptoms, at a sufficient concentration and for a time sufficient to elicit such an amelioration of myopia symptoms in the exposed animals. The response of the animals to the exposure may be monitored by assessing the reversal of characteristics, signs, or symptoms associated with myopia. With regard to intervention, any treatments which reverse any aspect of myopia-like characteristics, signs or symptoms should be considered as candidates for human myopia therapeutic intervention. Dosages of test agents may be determined by deriving dose-response curves.

Section 4. Pharmaceutical Compositions

The nicotinic antagonists of this invention have been found to possess valuable pharmacological properties. Nicotinic antagonists regulate postnatal growth of the eye, with the particularly desirable effect of inhibiting postnatal ocular growth and preventing the development of myopia. This effect can be demonstrated, for example, using the methods described in the Examples below.

Thus, these compounds can be used to control postnatal growth of the eye, inhibit postnatal ocular growth, prevent myopia, control abnormal postnatal ocular growth, inhibit abnormal postnatal axial growth of the eye, inhibit abnormal equatorial expansion of the eye, inhibit vitreous cavity expansion, inhibit the progression of myopia, inhibit the onset of myopia, and reverse myopia. These compounds are particularly useful to inhibit the development of myopia.

The compounds of this invention are generally administered to animals, including but not limited to mammals including humans, as well as to birds, monotremes, reptiles, or fish.

The pharmacologically active compounds of this invention can be processed in accordance with conventional methods of galenic pharmacy to produce medicinal agents for administration to patients, e.g., mammals including humans. The compounds of this invention can be employed in admixture with conventional excipients, i.e., pharmaceutically acceptable organic or inorganic carrier substances suitable for parenteral, enteral (oral) or topical ocular application which do not deleteriously react with the active compounds. Suitable pharmaceutically acceptable carriers include but are not limited to water, salt solutions, alcohols, vegetable oils, benzyl alcohols, polyethylene glycols, gelatine, carbohydrates such as lactose, amylose or starch, magnesium stearate, talc, silicic acid, viscous paraffin, fatty acid monoglycerides and diglycerides, pentaerythritol fatty acid esters, hydroxymethylcellulose, polyvinyl pyrrolidone, or any other suitable carrier. The pharmaceutical preparations can be sterilized and if desired, mixed with auxiliary agents, e.g., lubricants, preservatives, stabilizers, wetting agents, emulsifiers, salts for influencing osmotic pressure, buffers, and the like which do not deleteriously react with the active compounds. They can also be combined where desired with other active agents, e.g. vitamins.

For parenteral application, particularly suitable are injectable, sterile solutions, perferably aqueous or oily solutions, as well as suspensions, emulsions, or implants.

For enteral application, particularly suitable are tablets, dragees, liquids, drops, suppositories, or capsules. A syrup, elixir, or the like can be used wherein a sweetened vehicle is employed.

For topical application, there are employed liquid to viscous to semi-solid or solid forms comprising a carrier compatible with topical application and having a dynamic viscosity that might be preferably greater than water. Suitable formulations include but are not limited to, solutions, suspensions, emulsions, creams, ointments, gels, powders, liniments, salves, aerosols, etc., which are, if desired, sterilized or mixed with auxiliary agents, e.g., preservatives, stabilizers, wetting agents, buffers or salts for influencing osmotic pressure, etc. For topical application, also suitable are sprayable aerosol preparations wherein the active ingredient, prefereably in combination with a solid or liquid inert carrier material, is packaged in a squeeze bottle or otherwise propelled through a vehicle capable of aerosolizing the preparation.

The compounds of the instant invention are useful in treating or preventing the development of myopia. Therapy to inhibit axial elongation or equatorial expansion, control postnatal growth of the eye, inhibit postnatal ocular growth, prevent myopia, control abnormal postnatal ocular growth, inhibit abnormal postnatal axial growth of the eye, inhibit abnormal equatorial expansion of the eye, inhibit vitreous cavity expansion, inhibit the progression of myopia, inhibit the onset of myopia, and reverse myopia, can be administered by the use of the agent in eye drops. Eye drops are typically made up at a concentration of active agent between about 0.005% and 10% in the ophthalmic medium, advantageously between about 0.01% and 5%, and preferably between about 0.1% and 2%. A pH of about 3.5 to 8.5, advantageously about 4.0 to 8.0, and preferably about 4.5 to 7.5, may be expected to be acceptable as an ophthalmic drop. Phosphate buffering is also common for eye drops, but other buffers can be used. A common regimen for application of eye drops is one to four times a day spaced evenly throughout waking hours. More effective agents may require fewer applications or enable the use of more dilute solutions.

It will be appreciated that the actual preferred amounts of active compounds in a specific case will vary according to the specific compound being utilized, the particular compositions formulated, the mode of application, and the particular situs and organism being treated. Dosages for a given host can be determined using conventional considerations, e.g., by customary comparison of the differential activities of the subject compounds and of a known agents, e.g., by means of an appropriate, conventional pharmacological protocol.

### EXAMPLES

Methods. One-day-old white leghorn chicks (Truslow Farms, Chestertown, Md.) were reared in brooders on a 12 hour light-dark cycle with General Electric chroma 50 fluorescent lighting with irradiance of approximately 50 μW/cm² at chick eye level. The chicks received Purina Chick Chowg food and water ad libitum.

Experiments started at one week of age. For some chicks, a unilateral translucent white plastic goggle was glued to the periorbital feathers with cyanoacrylate glue to induce form-deprivation myopia, a commonly studied experimental model that induces ipsilateral myopia in newly hatched chicks and in those nearing maturity (Papastergiou et al., 1998, Vision Res., 38:1883-8). Under aseptic conditions, the goggled eye received a 10 μl intravitreal injection of either drug or saline vehicle at that time. Other chicks were non-goggled but similarly received intravitreal injections of either drug or vehicle to one eye. In most experimental groups, drug and vehicle were administered by intraocular injections daily or every other day at approximately four hours into the light phase. In each series, the experimental eye was alternated between left and right, and all contralateral eyes received injections of saline vehicle at the same time as injections to the experimental eye. Chicks were anesthetized with inhalation ether for all goggle applications and drug injections.

After one week of treatment and at two weeks of age, the chicks were anesthetized with an intramuscular mixture of ketamine (20 mg/kg) and xylazine (5 mg/kg), and ocular refractometry and A-scan ultrasonography were performed as described (Stone et al., 1995, Vision Res., 35:1195-202). No intraocular injections were administered on the day of examination. While still under general anesthesia, the chicks were decapitated and the axial and equatorial dimensions of enucleated eyes were measured with digital calipers. The coronal profile of the chick eye is elliptical, and the equatorial diameter is reported as the mean of the shortest and longest equatorial dimensions of the eye.

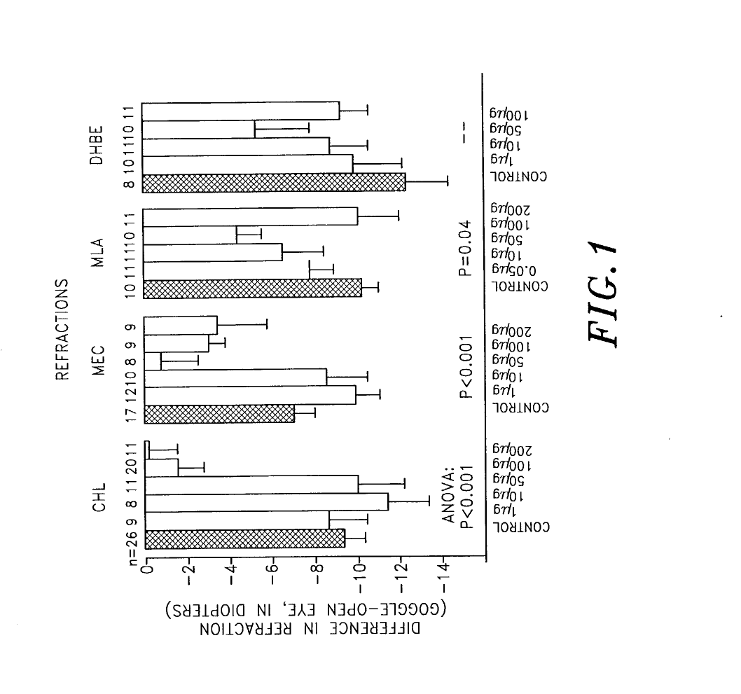

Data are provided as mean±S.E.M. and were analyzed with SigmaStat (SPSS, Inc. Chicago, Ill.). Neither visual deprivation or drug treatments to these eyes affected lens thickness, and these data are not reported for goggled chicks. A one-way analysis of variance (ANOVA), using the differences between visually deprived and contralateral eyes on goggled chicks, was performed to ascertain drug efficacy against experimental myopia. Because the ultrasound data on axial length following mecamylamine treatment to goggled eyes did not meet conditions of normality, these data were assessed with a Kruskal-Wallis one-way ANOVA on ranks on the differences between experimental and contralateral eyes. Data from different cohorts of chicks tested with the same drug, along with the respective vehicle-treated controls, were pooled for analysis (FIG. 1). Because the drug effects in the never-goggled chicks also were not normally distributed, drug treated non-goggled eyes and vehicle treated contralateral eyes were compared with a Friedman repeated measures ANOVA on ranks. In series when the ANOVA identified a treatment effect, post hoc multiple pairwise comparisons of the treatment groups were made with the Tukey test, using a value of P<0.05 for statistical significance. In assessing acute drug effects on ocular refractions and ultrasounds, the measurements before and after drug injection were compared with a Student's paired t-test. The experiments conformed with the ARVO Resolution on the Use of Animals in Ophthalmic and Vision Research.

### Example 1

The following drugs were administered daily: dihydro-β-crythroidine hydrobromide (RBI/Sigma; Natick, Mass.), mecamylamine (RBI/Sigma) and methyllcaconitine citrate (RBI/Sigma). Because it is a long-acting nicotinic antagonist in mammalian brain (El-Bizri and Clarke, 1994, Br. J. Pharmacol., 113:917-925), chlorisondamine diiodide (Tocris Cookson; Ballwin, Mo.) was generally administered every other day by intravitreal injection in most experiments.

Goggled chicks As expected from previous studies, the cohorts of vehicle treated control chicks wearing a unilateral goggle developed ipsilateral myopia of about −7 to −12 diopters compared to the contralateral non-goggled eyes. The axial lengths in the goggled eyes were increased by some 0.4-0.6 mm compared to the contralateral eyes. In general, the axial length difference between goggled and open eyes was greater as measured by ultrasound which records to the inner limiting membrane than as measured by calipers which records to the outer scleral surface. Besides the greater variability of the caliper measurements, this disparity may at least partly be physiologic as both the choroid and retina of young chicks thins during goggle wear. The vitreous cavity of goggled eyes was enlarged in both the axial and equatorial dimensions, with the vitreous cavity elongation largely accounting for the increase in overall axial length of the eye. Goggle wearing alone induced no significant effect on anterior chamber depth in most cohorts of vehicle treated chicks.

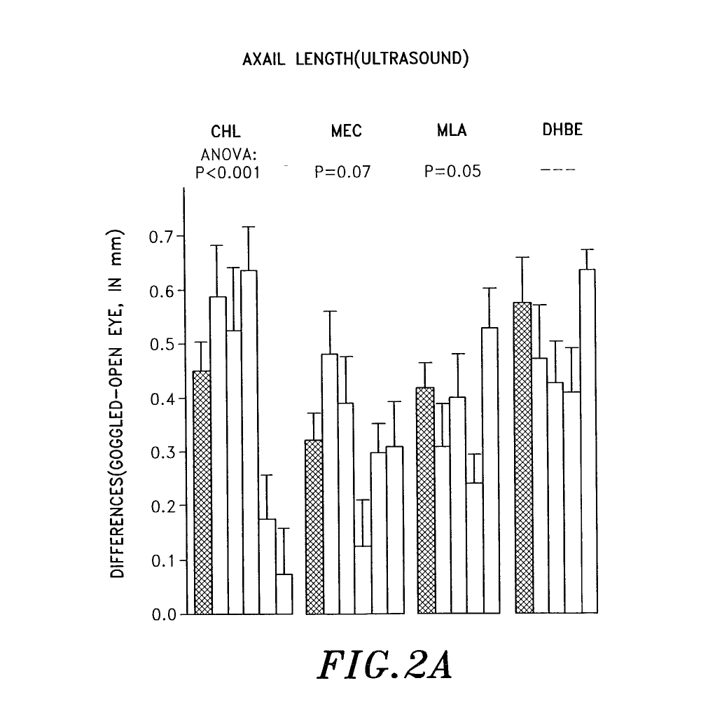

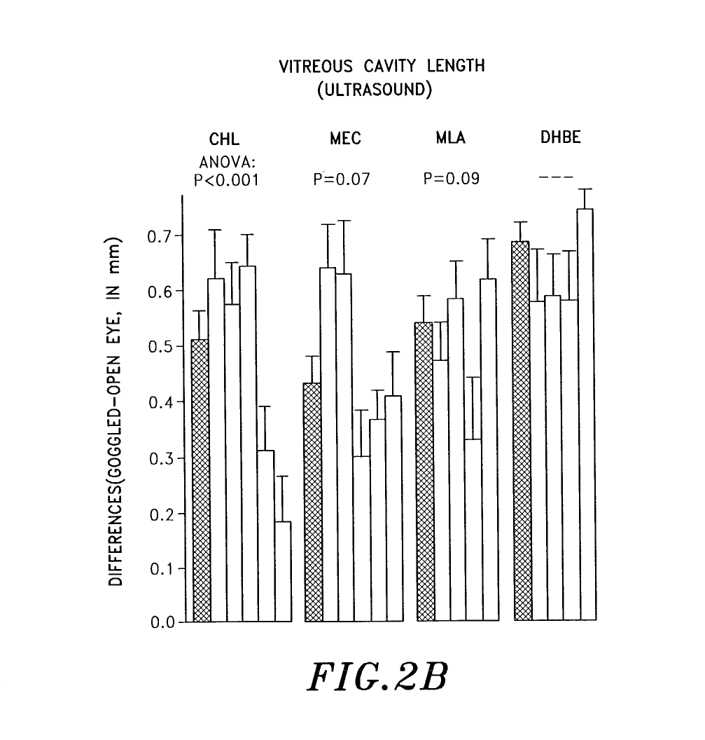

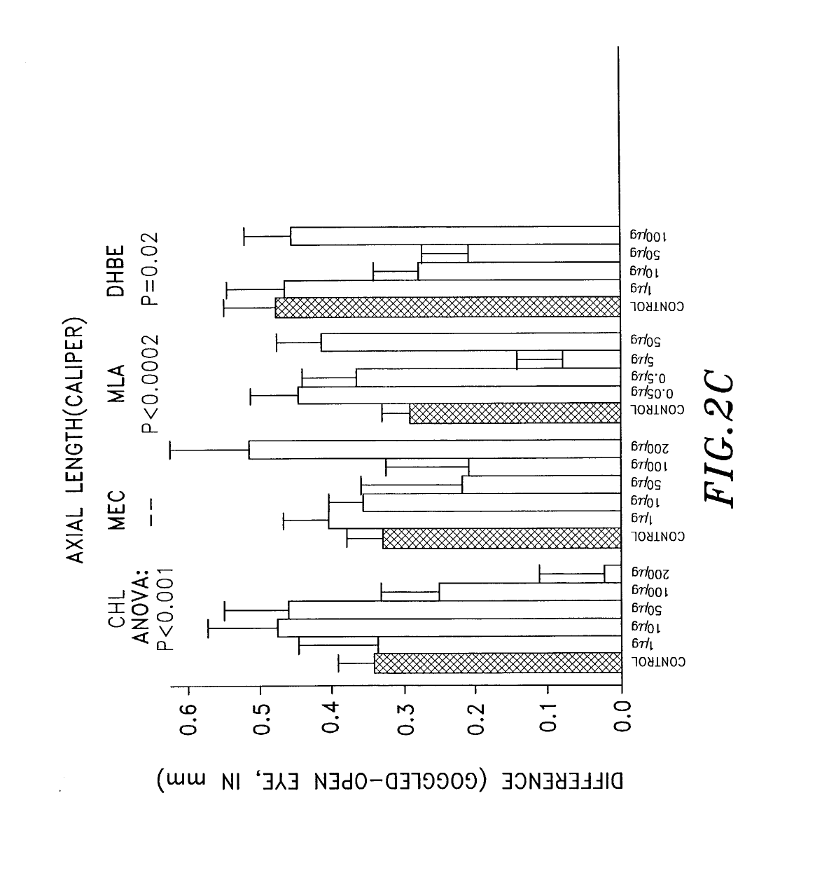

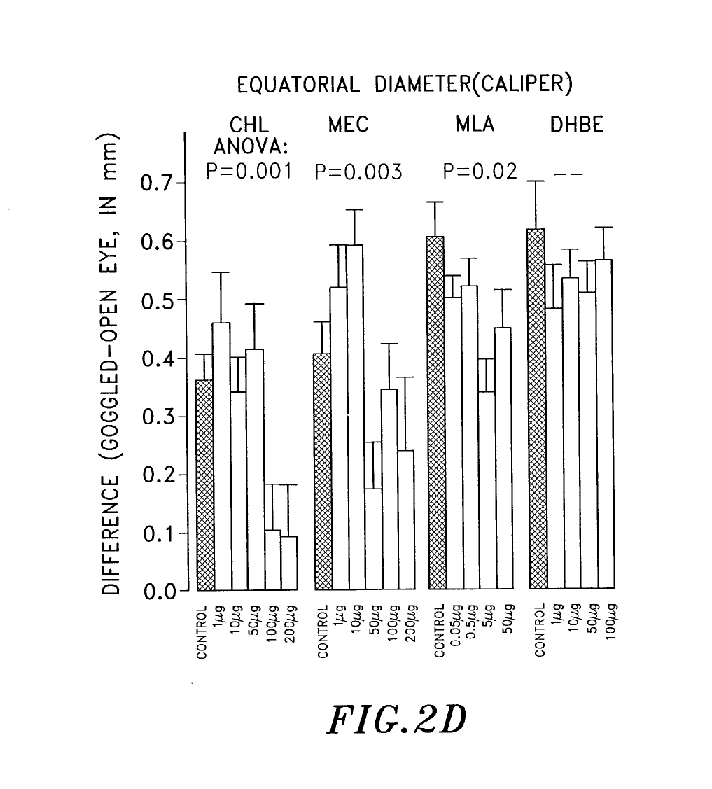

Two relatively non-selective nicotinic antagonists were tested, chlorisondamine and mecamylamine. Chlorisondamine reduced the myopic refractive error (FIG. 1; ANOVA: P<0.001), inhibited the excessive axial elongation developing beneath a goggle (FIGS. 2A, 2B, and 2C ANOVA: ultrasound, P<0.001; calipers, P=0.008) and reduced the vitreous cavity expansion in both axial (ANOVA: P<0.001) and equatorial (ANOVA: P=0.001) dimensions. Chlorisondamine had no statistically significant effect on anterior chamber depth. Post hoc pairwise comparisons by the Tukey test (Table 1) showed significant drug effects compared to the vehicle-treated controls for refraction, axial length and vitreous cavity depth measurements and for several other intragroup comparisons.

The effects of mecamylamine on goggled eyes were more complex, with a multiphasic response that differed between the low and high drug doses. It had a maximal anti-myopia effect at the intermediate dose and tended toward stimulating the growth and myopic refractive shift of goggled eyes at the lowest doses. Overall, mecamylamine altered refraction of goggled eyes (FIG. 1; ANOVA: P<0.001). Although all three higher drug doses reduced the induced myopia, only the 50 μg dose differed significantly from the controls by post hoc pairwise comparison testing (Table 1) and virtually eliminated the induced myopia. While the refractions at the 1 and 10 μg doses were not individually different from the controls by post hoc pairwise comparisons, significant differences occurred between the 1 μg dose and each of the 50, 100 and 200 μg doses as well as between the 10 and 50 μg doses (Table 1). The anatomical effects of mecamylamine on goggled eyes tended to follow the refractive effects: larger eyes developing with doses that did not reduce the myopia and smaller eyes with doses that did (FIG. 2). For axial length (FIGS. 2A and 2C), there was only a trend towards a drug effect by ultrasound (FIG. 2A ANOVA: P=0.07); no statistical effect on axial length by caliper measurements was apparent. Mecamylamine influenced the ultrasound measurements of vitreous cavity length (FIG. 2B ANOVA: P=0.007); post hoc pairwise comparison testing identified the 1 μg dose as different from the 50 μg dose but not from the controls (Table 1). Similarly, overall equatorial diameter of goggled eyes was influenced by mecamylamine (FIG. 2D ANOVA: P=0.003); post hoc pairwise comparison testing did not identify any individual doses that differed from the controls but showed that the 1 and 50 μg doses differed from each other and that the 10 μg dose differed from both the 50 and 200 μg doses (Table 1).

There also may have been an anterior chamber affect from mecamylamine (ANOVA: P=0.009), but post hoc pairwise comparison testing did not identify any individual differences. In reviewing the data on anterior chamber depth, the vehicle treated goggled eyes in the mecamylamine experiments had an anterior chamber depth slightly shallower than the contralateral non-goggled eyes (1.22±0.04 mm in goggled eyes versus 1.34±0.04 mm in non-goggled eyes). The differences in anterior chamber depth between goggled and contralateral eyes were similar for the low mecamylamine doses; but for the 100 and 200 μg doses, the anterior chamber depths in the drug treated goggled eyes relative to the contralateral controls were no longer reduced but instead were equal.

Of the antagonists with some subtype selectivity, methyllycaconitine showed the greater efficacy of the two drugs and was similar to mecamylamine in that the strongest effects seemed to occur at the intermediate drug doses. Methyllycaconitine affected the myopic refraction (ANOVA: P=0.04), axial length (ANOVA: ultrasound, P=0.05 (FIG. 2A); calipers, P=0.002 (FIG. 2C)), and equatorial expansion of the vitreous cavity (ANOVA: P=0.02 (FIG. 2D) in goggled eyes (FIGS. 1, 2). A trend towards an influence on vitreous cavity length did not reach significance with this drug (FIG. 2B ANOVA: P=0.09). With post hoc pairwise multiple comparisons by the Tukey test, a significant difference from controls was only identified for the inhibition of equatorial expansion beneath a goggle at the 5 μg dose; additionally, the 5 μg dose reduced axial length by calipers compared to the 0.05, 0.5 and 50 μg doses (Table 1). There was no effect from methyllycaconitine on the anterior chamber depth of goggled eyes.

Dihydro-β-erythroidine exhibited only a weak effect against experimental myopia (FIGS. 1, 2). The drug induced a significant reduction only in axial length as measured by calipers (ANOVA: P=0.02 (FIG. 2C), but no individual drug dose was identified by the post hoc pairwise multiple comparison testing. Otherwise, none of the differences in refraction, ultrasound measurements or caliper measurements of the equatorial diameter reached statistical significance by ANOVA.

Non-goggled chicks. Chicks were reared and treated as described in the Methods section above, but without goggles. Under aseptic conditions, one non-goggled, or “open” eye received a 10 μl intravitreal injection of either drug or saline vehicle. All contralateral eyes received injections of saline vehicle at the same time as injections to the experimental eye. Drug and vehicle were administered by intraocular injections daily or every other day at approximately four hours into the light phase.

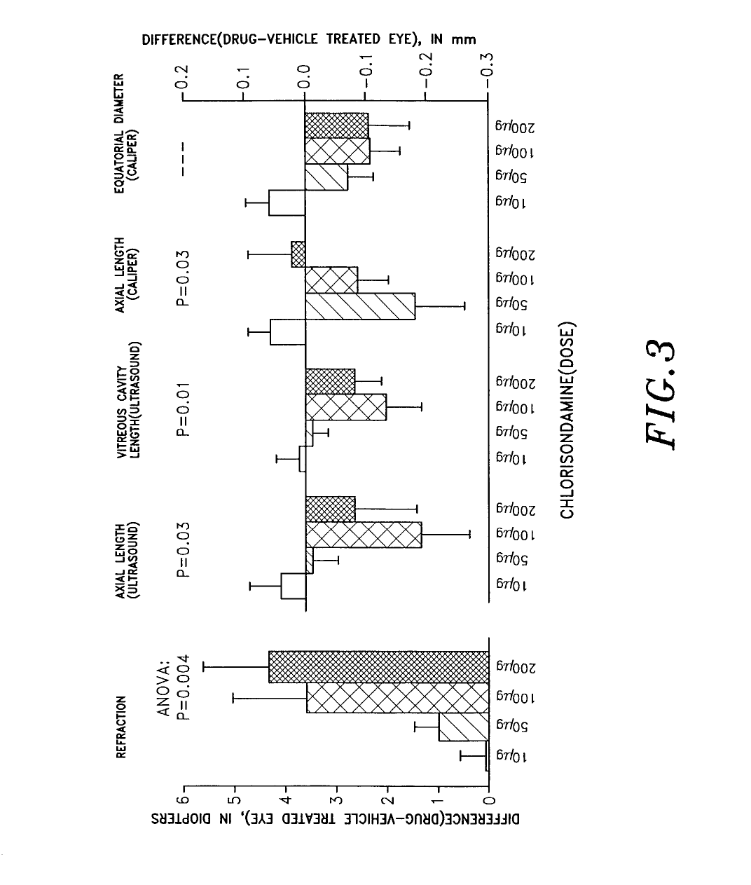

Unilateral intravitreal administration of chlorisondamine reduced the axial growth of drug-treated eyes in never-goggled chicks (FIG. 3; ANOVA on ranks: ultrasound, P=0.03; calipers, P=0.03). The growth reduction was confined to the vitreous cavity (ANOVA on ranks: P=0.01) and was reflected in a hyperopic shift in refraction (ANOVA on ranks: P=0.004). The effect on equatorial expansion of the vitreous cavity did not reach statistical significance. Pairwise comparisons with the Tukey test identified the refractions of the eyes treated with 200 μg and 10 μg and the vitreous cavity depths of the eyes treated with 100 μg and 50 μg as different from each other (Table 1). An effect on lens thickness also was noted (ANOVA on ranks: P<0.001), comprising an increase of about 0.1 mm in both eyes in the 10 μg group compared to chicks receiving the 50, 100 or 200 μg doses as well as other chicks who received saline injections to both eyes; no pairwise comparisons of the lenses were identified as significant by the Tukey test, however.

In contrast to the chlorisondamine effects on open eyes, daily intravitreal injections of mecamylamine had no influence on the growth or refraction of non-goggled eyes after one week at doses of 50 μg (the dose with the strongest effect against form-deprivation myopia) or of either 10 or 1 μg (the doses that tended to stimulate the myopic response to a goggle).

### Example 2

Acute drug effects. To assess acute drug effects, other two-week old chicks (n=5/group) received a single unilateral intravitreal injection of one of the nicotinic antagonists at doses chosen based on drug effects on the growth of goggled eyes: 200 μg chlorisondamine, 50 μg and 1 μg mecamylamine, 5 μg methyllycaconitine or 50%g dihydro-β-erythroidine. Just before and at two hours and 24 hours after injection, both eyes were examined by refractometry and ultrasonography by the above methods. Because chicks in the eye growth studies did not receive drug on the day of measurements, the 24 hour examination point was selected specifically to identify a potential residual drug effect on the intraocular muscles at a time relevant to the eye growth measurements.

Mean baseline pupil diameter measured 2.4±0.5 mm. Two hours after injection, each of the drugs induced some pupillary dilation (change from baseline: 200 μg chlorisondamine, 0.8±0.1 mm, P<0.01; 50 μg mecamylamine, 0.3±0.1 mm, not significantly changed; 1 μg mecamylamine, 0.8±0.2 mm, P<0.05; 5 μg methyllycaconitine 0.4±0.2 mm, not significantly changed; 50 μg dihydro-β-erythroidine 1.0±0.1 mm, P<0.01). Although dilated, the pupils in each group still constricted in response to light but were sluggish. By 24 hours, the pupil had returned to normal in all but two groups (change from baseline: chlorisondamine, 0.7±0.2 mm, P<0.05; mecamylamine 50 μg, 0.4±0.1 mm, P<0.05). None of the drug applications had a significant effect on refraction at either 2 or 24 hours. By ultrasonography, chlorisondamine induced a 0.16±0.04 mm (P<0.05) reduction in axial length and a 0.20±0.07 mm (P<0.05) reduction in posterior chamber depth at 2 hours, each of which returned to baseline at 24 hours; chlorisondamine also reduced lens thickness by 0.12±0.04 mm (P<0.05) at 2 hours and by 0.16±0.05 mm (P<0.05) at 24 hours. None of the other drugs influenced the ultrasound measurements.

In these experiments, the eyes were examined 2 and 24 hours after administration of selected doses of nicotinic antagonists to learn if any acutely influenced the tone of the intraocular muscles. Each drug induced some mydriasis (pupil dilation), reversible to light; presumably any cycloplegic (inhibition of accommodation) effect also was partial. Based on the results, these nicotinic antagonist drugs neither shifted refraction acutely nor uncovered any basal accommodative tone under the conditions of the examinations. Only chlorisondamine acutely altered ocular dimensions by ultrasound, transiently reducing axial and vitreous cavity lengths at the 2 hour but not at the 24 hour reading. Further, only chlorisondamine influenced lens thickness, reducing it and likely increasing the focal length; if any increase in lens focal length had modulated development of open eyes receiving this drug daily, it would have stimulated eye growth (Schaeffel F, Glasser A, Howland HC, 1988, Vision Res., 28:639-657) and not inhibited it (FIG. 3). Because all measurements of drug influences on eye growth were made 24 hours after the last dosing, none of the observations on growth or refractive development shown in Examples 1 and 3 can be explained by an acute drug effect, muscular or otherwise, on refraction or eye component measurements; that is, the effects of nicotinic antagonists on ocular refraction and eye growth (Examples 1 and 3) are effects on the development of the eye and constitute developmental modification of ocular refraction and/or dimensions.

### Example 3

Longer term effects of single-dose chlorisondamine. In a separate experiment, another group of goggled chicks received a single intravitreal dose of 200 μg of chlorisondamine to the form deprived eye and saline to the contralateral eye only at the time of goggle application. They were compared to a group of unilaterally goggled chicks receiving a single saline injection to both eyes only at the time of goggle application. Neither group received subsequent intraocular injections. These chicks were evaluated by refractometry and ultrasound 4 days later, using ketamine/xylazine anesthesia. At one week after goggle application and single drug injection, the same chicks were evaluated again with refractometry, ultrasonography and caliper measurements of enucleated eyes as described above.

A single dose of 200 μg of chlorisondamine given at the time of goggle application significantly blunted the response for form deprivation over the next week (Table 2). The effect is manifest at four days by significant attenuations in refraction (p<0.05) and axial length (p<0.01) by ultrasonography of goggled eyes. While the effectiveness against the myopic shift in refraction is diminished at one week, statistically significant attenuations are evident at one week in the ultrasound measurements of axial length (p<0.05) and vitreous cavity depth (p=0.05) and in the caliper measurements of axial length (p<0.05) and equatorial diameter (p<0.005). The action of a single dose of chlorisodamine against myopic eye growth seems equivalent at four days and one week because the ratios of axial and vitreous cavity lengths of the drug treated to vehicle treated eyes are equivalent at both times (Table 2).

### Example 4

Histopathological effects. To identify potential histopathologic effects in other groups of monocularly deprived or never-goggled one-week old chicks, chlorisondamine (200 or 100 μg every other day; n=5-6/group), mecamylamine (200 or 50 μg daily; n=5-8/group) or saline vehicle (n=3-9/group) was administered by intravitreal injection to the goggled eyes or to one of the open eyes of never-goggled chicks with vehicle to the contralateral eye, using the identical protocol as above. After one week of treatment, the above protocols provided refraction, ultrasound and caliper measurements. The eyes were then immersion fixed in 3% glutaraldehyde/0.5% paraformaldehyde in 0.1M phosphate buffer, pH 7.4. The posterior segments were either embedded in paraffin, cut at 5 μm thickness and stained with hematoxylin and eosin, or embedded in historesin, cut at 3 μm or 5 μm thickness and stained with 0.5% azure II/0.5% methylene blue in 1% borate.

With chlorisondamine 200 μg every other day, gross inspection of the eye cup of most of the goggled eyes (4/5) and all of the never-goggled eyes (n=5) showed mild to marked mottling and depigmentation of the mid-peripheral fundus; a variably sized geographic area appeared relatively spared or normal in the central fundus region. The tissue sections revealed marked disruption and clumping of cells of the retinal pigment epithelium (RPE) in regions corresponding to the peripheral depigmented areas. Pigment containing cells, presumably macrophages, were occasionally noted in the outer retina, and outer segments were sometimes disrupted overlying the disrupted epithelium. The retina otherwise appeared intact. Presumed inflammatory cells infiltrated the peripheral choriocapillaris beneath the most involved areas of the RPE, but the choroid was otherwise unaffected. The central regions of these eyes showed either normal histology or less marked changes. The goggled eye treated with 200 μg chlorisondamine that had a normal gross examination also exhibited normal histology. Of the goggled or non-goggled eyes treated with chlorisondamine 100 μg every other day, gross inspection of the eye cups showed either a normal fundus or only mild peripheral pigmentary changes. Some eyes had normal histology, some showed a single large, smooth hyper-pigmented inclusion within a rare RPE cell as the sole detectable histologic change, and some had a small isolated peripheral patch of the marked RPE/choriocapillaris pathology as described above. Importantly, the growth and refractive responses of goggled or open eyes to chlorisondamine was not clearly related to the degree of retinal histopathology.

The retinas of goggled and non-goggled eyes treated daily with either 200 or 50 μg of mecamylamine were indistinguishable grossly or histologically from vehicle treated control eyes.

All of the references cited herein, whether research articles, patent documents, or other cited references, are hereby incorporated by reference in their entirety as though individually incorporated by reference. It can be understood by those of skill in the art that numerous and various modifications can be made without departing from the spirit of the present invention. Therefore, it should be clearly understood that the forms of the present invention are illustrative only and are not intended to limit the scope of the present invention.

TABLE A
Nicotinic Receptors
Acetylcholine Receptors (Nicotinic)

| Nomenclature* | α1* | α2* | α3* | α4* |
|---|---|---|---|---|
| Natural location(s) | muscle | CNS | ANS, CNS | CNS, sensory ganglia |
| Agonists† | iso; sub; ACh; | ABT418^{b}; nicotine^{c}; | cytisine^{a}, epibatidine^{d}; | cytisine^{a}, ABT418^{b}, |
| | epibatidine; BAC; | epibatidine^{d}; DMPP; | DMPP; nic; AUh; ABT- | nicotine^{c}, epibatidine^{d}; |
| | DMAP; DMPP; | anatoxin; cytisine; | 418; anatoxin; DMPP; | anatoxin; DMPP; ACh |
| | carbamylcholine; suc; | ACh; sub | sub; iso; BAC; | |
| | cytisine; nicotine | | carbamylcholine; | |
| | | | DMAP; suc | |
| Selective competitive | α-bungarotoxin, | dihydro-β-erythroidine | dihydro-β-erythroidine | dihydro-β-erythroidine |
| antagonists | dTC [high (α1/γ) and | | | |
| | low (α1/δ) affinity sites] | | | |
| Other competitive | α-conotoxins GI & MI; | dTC | α-conotoxin MII; | methyllycaconitine; |
| antagonists | alcuronium; nstx; trim; | | α-conotoxin AulB; | alcuronium; dTC; eser; |
| | ben; mecamylamine; | | decamethonium | decamethonium |
| | lobeline; eser; | | | |
| | decamethonium; | | | |
| | atropine; gallamine; | | | |
| | hemicholinium' | | | |
| | strychnine; neostigmine; | | | |
| | dihydro-β-erythroidine; | | | |
| | hexamethonium; | | | |
| | choline | | | |
| Noncompetitive | gallamine | hexamethonium, | hexamethonium, | hexamethonium, |
| channel blocker | | mecamylamine | mecamylamine | mecamylamine |
| antagonists | | | chlorisondamine; | |
| Radioligands | [³H] or [¹²⁵I]- | [³H]-acetylcholine | [³H]-acetylcholine | [³H]-acetylcholine |
| | bungarotoxin (ACh) | (ACh) | (ACh) | |
| | | [³H]-cytisine | [³H]-cytisine | [³H]-cytisine |
| | | [³H]-epibatidine | [³H]-epibatidine | [³H]-epibatidine |
| | | [³H]-methylcarbamyl- | [³H]-methylcarbamyl- | [³H]-methylcarbamyl-choline |
| | | choline | choline | [³H]-nicotine |
| | | [³H]-nicotine | [³H]-nicotine | |
| Effector | Int cat. (P_{Ca}/P_{Na}0.2-1) | Int. cat. (Na⁺, Ca²⁺; | Int. cat. (Na⁺, Ca²⁺; | Int. cat (P_{Ca}/P_{Na}0.5-6) |
| | | P_{Ca}/P_{Na}) | P_{Ca}/P_{Na}) | |
| Structural | | | | |
| Information | | | | |
| Receptor subtypes | (α1)₂β1γδ-embryonic | α2β2^{b}, α2β4 | α3*, α3β4*, α3α5β4, | α4*, (α4)₂(β2)₃ |
| identified, in vivo | (α1)₂β1 δ-adult | | α3α5β2β4, α3β23β3β4 | α4α5β2 |
| Functional receptor | (α1)₂β1γδ, (α1)₂β1εδ | α2/β2, α2/β4 | α3β2^{d}, α3β4^{a}, | α4β2^{a,b,c,d}, α4β4^{a,c} |
| subtypes created by | α1β2γδ, α1β4γδ | | α3α5β2, α3α5β4, | α4α5β2, α4β2β4 |
| heterologous expression | | | α3β3β4 | α4β2β3β4 |
| Binding site interfaces | α1/γ or α1/ε and α1/δ | | α3/β2^{d}, α3/β4^{a} | α4/β2, α4/β4 |
| | | | α8 (avian), α8* | |
| Nomenclature* | α6* | α7, α7* | (avian) | α9, α9* |
| Natural location(s) | CNS | ANS, CNS | retina, CNS | pituitary, cochlea |
| Agonists† | epibatidine^{d;} | anatoxin, choline¹⁻³ | cytisine; nicotine; ACh; | ACh |
| | cytisine; DMPP, | DMAC4; | DMPP; TMA | |
| | nicotine, ACh | epibatidine; OH-GTS- | | |
| | | 21; DMPP; cytisine; | | |
| | | nicotine; GTS-21; ABT- | | |
| | | 418; ACh, TMA, | | |
| | | carbamylcholine | | |
| Selective antagonists | dihydro-β-erythroidine | α-bungarotoxin, | α-bungarotoxin | α-bungarotoxin, nicotine, |
| | | methyllycaconitine | | |
| Other antagonists | dTC | α-conotoxin IMl; α- | atropine; dTC; | strychnine, atropine; |
| | | bungarotoxin; dTC; | strychnine | dTC; muscarine |
| | | atropine; dihydro-β- | | |
| | | erythroidine | | |
| Channel blocker | hexamethonium, | — | — | — |
| | mecamylamine | | | |
| Radioligands | — | [³H]/[I¹²⁵I]- | [³H]/[¹²⁵I]-bungarotoxin | — |
| | | bungarotoxin | | |
| Effector | Int. cat. (Na⁺, Ca²⁺; | Int. cat. (P_{Ca}/P_{Na}6-20) | Int cat (P_{Ca}/P_{Na}) | Int cat (Na⁺, Ca²⁺) |
| | P_{Ca}/P_{Na}) | | | |
| Structural | | | | |
| Information | | | | |
| Receptor subtypes | α6α3β2, α6α3β4, | (α7)₅, α7α8, α7* | (α8)₅, α7α8 | α9* |
| identified, in vivo | α6β2β4, | | | |
| | α6/β4^{d} | | | |
| Functional receptor | α6α3β2, α6α4β2 | (α7)₅, α7/α8 | (α8)₅, α7α8 | (α9)₅, α9α10 |
| subtypes created by | α6β3β4, α6β4 | | | |
| heterologous expression | | | | |
| Binding site interfaces | α6/β2, α6β4 | α7/α7, α7/α8 (avian) | α8/α8, α8/α7 | α9/α9 (not α10₅) |

†Superscripts (a,b,c and d) indicate ligands showing some selectivity for the binding site interfaces, similarly labelled
Chemical names:
ABT418, (S)-3-methyl-5-(1-methyl-2-pyrrolidinyl) isoxazole;
ACh, acetylcholine;
ben, benzoquinonium;
BAC, bromoacetylcholine;
DMAB, 3-(4)-dimethylaminobenzylidine anabaseine;
DMAC, 3-(4)-dimethylaminocinnamylidine;
DMAP, N-N-dimethyl-4-acetylpiperzinium;
DMPP, 1,1-dimethyl-4-phenylpiperazinium;
dTC, d-tubocurarine, or (+)-tubocurarine;
eser, eser(physostigmine);
GTS-21, 3-(2,4)-diemthoxybenzylidine anabaseine (DMXB);
iso, isoarecolone methiodide;
nstx, neosurugatoxin; OH-GTS-21, 3-(4-hydroxy, 2-methoxy)benzylidine anabaseine;
sub, suberyldicholine;
suc, succinyl(di)choline;
TMA, tetramethylammonium;
trim, trimethaphan.

TABLE 1
Post Hoc Pairwise Comparisons of Drug Effects Using Tukey Test

| | | Ultrasound | | Caliper | |
|---|---|---|---|---|---|
| Condition and | | measurements | vitreous cavity | measurements | equatorial |
| drug | refraction | axial length | depth | axial length | diameter |
| Goggled chicks | | | | | |
| chlorisondamine | 200 μg vs. | 200 μg vs. | 200 μg vs. | 200 μg vs. | 200 μg vs. |
| | control, 50, 10 | control, 50, 10 | control, 50, 10 & | control, 50 & | 1 μg |
| | & 1 μg | & 1 μg | 1 μg | 10 μg | 100 μg vs. |
| | 100 μg vs. | 100 μg vs. 50 & | 100 μg vs. 50 μg | | 1 μg |
| | control, 50, 10 | 1 μg | | | |
| | & 1 μg | | | | |
| mecamylamine | 200 μg vs. 1 μg | — | 50 μg vs. 1 μg | — | 200 μg vs. |
| | 100 μg vs. 1 μg | | | | 10 μg |
| | 50 μg vs. | | | | 50 μg vs. 10 |
| | control, 10 & | | | | & 1 μg |
| | 1 μg | | | | |
| methyll-caconitine | — | — | — | 5 μg vs. 50, 0.5 | 5 μg vs. |
| | | | | & 0.05 μg | control |
| dihydro-β- | — | — | — | — | — |
| erythroidine | | | | | |
| Non-goggled | | | | | |
| chicks | | | | | |
| chlorisondamine | 200 μg vs. 10 μg | — | 100 μg vs. 50 μg | — | — |
| treated eyes | | | | | |

The statistically significant (defined as P<0.05) post hoc pairwise comparisons by the Tukey test are shown for each condition and drug for which the ANOVA identified a treatment effect (see text and FIGS. 1-3).

TABLE 2
Longer Term Effects of Single-dose Chlorisondamine (200 μg) on
Form Deprivation Myopia.

| Time After Drug Administration | |
|---|---|
| 4 Days | 1 Week |

Refraction Difference

| | | | |
|---|---|---|---|
| vehicle | n = 7 | −8.82 ± 1.07 diopters | −11.72 ± 1.19 diopters |
| chlorisondamine | n = 10 | −2.98 ± 1.88 diopters** | −8.58 ± 2.06 diopters |

Ultrasound Measurements
axial length difference

| | | |
|---|---|---|
| vehicle | 0.43 ± 0.04 mm | 0.61 ± 0.06 mm |
| chlorisondamine | 0.20 ± 0.07 mm*** | 0.33 ± 0.11 mm** |
| ratio-chlorisondamine/vehicle | 0.47 | 0.54 |
| vitreous cavity length difference | | |
| vehicle | 0.40 ± 0.04 mm | 0.69 ± 0.06 mm |
| chlorisondamine | 0.28 ± 0.06 mm | 0.49 ± 0.08 mm* |
| ratio-chlorisondamine/vehicle | 0.70 | 0.71 |
| Caliper Measurements | | |
| axial length difference | | |
| vehicle | — | 0.51 ± 0.06 mm |
| chlorisondamine | — | 0.22 ± 0.10 mm** |
| equatorial diameter difference | | |
| vehicle | — | 0.51 ± 0.06 mm |
| chlorisondamine | — | 0.17 ± 0.07 mm**** |

*p = 0.05; **p < 0.05; ***p < 0.01; ****p < 0.005, comparing the drug treated to vehicle treated groups, using one-way analysis of variance on the difference of goggled and contralateral eyes. The number of chicks in each group are shown with the refractions.

What is claimed is:

1. A method of controlling postnatal ocular growth comprising the step of:
ocular administration of a therapeutically effective amount of a nicotinic antagonist to control postnatal ocular growth.

2. A method of inhibiting the abnormal axial growth of the eye of a host animal comprising the step of:
administering to said eye during postnatal development a therapeutically effective amount of a nicotinic antagonist to inhibit the abnormal postnatal axial growth of the eye.

3. A method of inhibiting the abnormal equatorial expansion of the eye of a host animal comprising the step of:
administering to said eye during postnatal development a therapeutically effective amount of a nicotinic antagonist to inhibit the abnormal equatorial expansion of the eye.

4. A method of inhibiting the abnormal vitreous cavity expansion of the eye of a host animal comprising the step of:
administering to said eye during postnatal development a therapeutically effective amount of a nicotinic antagonist to inhibit the abnormal vitreous cavity expansion of the eye.

5. A method of inhibiting development of myopia comprising the step of:
ocular administration of a therapeutically effective amount of a nicotinic antagonist to inhibit development of myopia.

6. The method according to any of claims 1 or 5, wherein said nicotinic antagonist is a competitive nicotinic antagonist.

7. The method according to claim 6, wherein said competitive nicotinic antagonist is methyllcaconitine.

8. The method according to claim 6, wherein said competitive nicotinic antagonist is dihydro-β-erythroidine.

9. The method according to any of claims 1 or 5, wherein said nicotinic antagonist is a channel-blocking nicotinic antagonist.

10. The method according to claim 9 wherein said channel blocking nicotinic antagonist is mecamylamine.

11. The method according to claim 9 wherein said channel blocking nicotinic antagonist is chlorisondamine.

12. The method according to any of claims 1 or 5, wherein said nicotinic antagonist is a noncompetitive nicotinic antagonist.

13. The method according to claim 12, where said noncompetitive nicotinic antagonist is selected from the group consisting of sertraline, paroxetine, nefaxodone, venlafaxine, fluoxetine, buproprion, phencyclidine, and ibogaine.

14. The method according to any of claim 1, wherein said nicotinic antagonist is an antibody inhibiting nicotinic receptor function.

15. The method according to any of claim 1, wherein said nicotinic antagonist is an agonist that acts like a nicotinic antagonist.

16. A method of detecting the ability of a nicotinic antagonist to control postnatal ocular growth of the eye of a host animal comprising the steps of:
contacting a first animal eye with a therapeutically effective amount of a nicotinic antagonist;
detecting the change in growth of said first animal eye;
applying a known control agent in a second animal eye;
observing the results of said control agent on the change in growth of said second eye; and
comparing the change in growth of said first eye with said change in growth of said second eye, thereby identifying the nicotinic antagonist as having the ability to control postnatal ocular growth.

17. A method of making a pharmaceutical comprising the steps of:
identifying a nicotinic antagonist as an active agent having the ability to control postnatal ocular growth and combining the active agent in admixture with a pharmaceutical excipient.

18. A method of identifying a compound that modulates myopia comprising the steps of:
(a) incubating a cell that expresses a nicotinic receptor in the presence and absence of a test compound;
(b) determining whether said test compound binds to said nicotinic receptor;
(c) selecting a test compound that binds to said nicotinic receptor;
(d) administering said selected test compound of step (c) to a test animal;
(e) determining whether said test compound alters the development of myopia of said test animal; and
(f) selecting a compound that alters said development of myopia of said test animal.
